(19) United States
(12) Patent Application Publication
(10) Pub. No.: US 2005/0031569 A1
(43) Pub. Date: Feb. 10, 2005

(54) Cosmetic and/or pharmaceutical preparations

(76) Inventors: Werner Seipel, Hilden (DE); Mirella Nalborczyk, Titz (DE); Claus Nieendick, Krefeld (DE); Anke Becker, Duesseldorf (DE)

Correspondence Address: COGNIS CORPORATION;PATENT DEPARTMENT, 300 BROOKSIDE AVENUE, AMBLER, PA, 19002, US

(21) Appl. No.: 10/492,317

(22) PCT Filed: Oct. 4, 2002
(86) PCT No.: PCT/EP02/11106

(30) Foreign Application Priority Data
Oct. 13, 2001 (DE) 101 50 730.5

(51) Int. Cl. A61K 7/075; A61K 7/08

(52) U.S. Cl. 424/70.21; 424/70.22; 424/70.24

(57) ABSTRACT

A cosmetic and/or pharmaceutical composition containing: (a) a fruit acid ester; (b) a surfactant selected from the group consisting of an anionic surfactant, a nonionic surfactant, a cationic surfactant, an amphoteric surfactant, a zwitterionic surfactant, and mixtures thereof; (c) a fatty alcohol; and (d) optionally, water.

### FIELD OF THE INVENTION

This invention relates to surfactant preparations containing fruit acid esters and fatty alcohols and to the use of fruit acid esters as lipid layer enhancers in surfactant preparations.

### PRIOR ART

Preparations used to clean and care for the human skin and hair generally contain one or more surfactants, more particularly based on anionic or amphoteric surfactants. Since the use of surfactants on their own would excessively dry out the skin and hair, lipid layer enhancers are generally added to such preparations. However, surfactant-containing formulations on the one hand are intended to have a cleaning effect, but on the other hand are nevertheless intended to leave a lipid-layer-enhancing protective film behind on the skin. With rinse-off formulations in particular, the difficulty is that lipid-layer-enhancing substances are also rinsed off and are unable adequately to develop their effect. The replacement of unwanted fatty constituents on the skin, which are intended to undergo the cleaning process, by lipid-layer-enhancing constituents which are intended to remain on the skin or to be incorporated in the upper layers of the skin is subject to a delicate balance.

U.S. Pat. No. 5,089,531 discloses salts of citric acid monoesters as skin-smoothing and moisturizing components. However, the lipid-layer-enhancing effect of these preparations is unsatisfactory. U.S. Pat. No. 6,024,947 relates to rinse-off formulations containing an oil phase and citric acid esters of which the effect in this case is to improve the rinse off behavior of the oil-containing formulation. In this formulation, the lipid-layer-enhancing properties are neglected in favor of the cleaning effect.

German patent DE 19621681 C2 relates to water-based pearlizing concentrates in which hydroxysubstituted carboxylic acid esters are used as the pearlizing component.

The problem addressed by the present invention was to provide surfactant formulations which would combine a good cleaning effect with an improved lipid-layer-enhancing effect in relation to the prior art and which would leave the skin with a pleasant feeling, would show high dermatological compatibility and would be easy to produce. The lipid layer enhancers used in the preparations would not reduce their viscosity.

### DESCRIPTION OF THE INVENTION

The present invention relates to cosmetic and/or pharmaceutical preparations containing
(a) 0.1 to 30% by weight fruit acid esters,
(b) 0.1 to 90% by weight anionic and/or nonionic and/or cationic and/or amphoteric and/or zwitterionic surfactants and
(c) 0.1 to 10% by weight fatty alcohols,
with the proviso that the quantities shown add up to 100% by weight with water and optionally typical auxiliaries and additives,
and to the use of fruit acid esters as lipid layer enhancers in surfactant formulations.

It has surprisingly been found that the combination of fruit acid esters with surfactants leads to rinse-off formulations which combine a very good cleaning effect with optimal lipid-layer-enhancing properties. The mixtures leave the skin with a pleasant feeling and show particularly high dermatological compatibility. Neither foam volume nor foam stability is reduced despite the favorable lipid-layer-enhancing effect. When used in hair-care preparations, the mixtures produce an improvement in combability and manageability and a pleasant soft feel. The processing behavior of the mixtures can be improved by the addition of fatty alcohols without any shift in the balance between cleaning effect and lipid-layer-enhancing effect, despite the change in the lipophilic/hydrophilic balance. The formulations can be processed without heat and their liquid consistency enables them to be uniformly applied.

Fruit Acid Esters

The fruit acid esters selected are known substances which may be prepared by the relevant methods of preparative organic chemistry. The fruit acid component is selected from citric acid, malic acid, tartaric acid, mandelic acid and/or racemic acid, although citric acid and malic acid esters are preferably used. The acid component is esterified with saturated and unsaturated, long-chain and branched alcohols with a chain length of 8 to 22 carbon atoms, preferably 16 to 18 carbon atoms and more particularly 18 carbon atoms. Depending on the number of acid functions, mono-, di and triesters and mixtures thereof may be used. Trialkyl and dialkyl esters are preferred, trialkyl esters of citric acid and dialkyl esters of malic acid being particularly preferred. Citric acid trioctadecyl ester and malic acid dioctadecyl ester are most particularly preferred. These fruit acid esters are used in the preparations according to the invention in quantities of 0.1 to 30% by weight, preferably in quantities of 0.5 to 25% by weight and more particularly in quantities of 1 to 20% by weight.

Surfactants

Suitable surfactants are nonionic, anionic, cationic and/or amphoteric or zwitterionic surfactants which are normally present in the preparations in quantities of about 0.1 to 90, preferably 5 to 70 and more particularly 10 to 50% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl(ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds and esterquats, more particularly quaternized fatty acid trialkanolamine ester salts. Typical examples of amphoteric or zwitterionic surfactants are alkylbetaines, alkylamido-betaines, aminopropionates, aminoglycinates, imidazolinium betaines and sulfobetaines. The surfactants mentioned are all known compounds. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Alkyl and/or Alkenyl Oligoglycosides

Alkyl and alkenyl oligoglycosides which are used as partiularly preferred surfactants are known nonionic surfactants which corresponding to formula (I):
R¹O-[G]ₚ (I)
in which R¹ is an alkyl and/or alkenyl group containing 4 to 22 carbon atoms, G is a sugar unit containing 5 or 6 carbon atoms and p is a number of 1 to 10. They may be obtained by the relevant methods of preparative organic chemistry, for example by acid-catalyzed acetalization of glucose with fatty alcohols.

The alkyl and/or alkenyl oligoglycosides may be derived from aldoses or ketoses containing 5 or 6 carbon atoms, preferably glucose. Accordingly, the preferred alkyl and/or alkenyl oligoglycosides are alkyl and/or alkenyl oligoglucosides. The index p in general formula (I) indicates the degree of oligomerization (DP), i.e. the distribution of mono- and oligoglycosides, and is a number of 1 to 10. Whereas p in a given compound must always be an integer and, above all, may assume a value of 1 to 6, the value p for a certain alkyl oligoglycoside is an analytically determined calculated quantity which is generally a broken number. Alkyl and/or alkenyl oligoglycosides having an average degree of oligomerization p of 1.1 to 3.0 are preferably used. Alkyl and/or alkenyl oligoglycosides having a degree of oligomerization of less than 1.7 and, more particularly, between 1.2 and 1.4 are preferred from the applicational point of view.

The alkyl or alkenyl group R¹ may be derived from primary alcohols containing 4 to 11 and preferably 8 to 10 carbon atoms. Typical examples are butanol, caproic alcohol, caprylic alcohol, capric alcohol and undecyl alcohol and the technical mixtures thereof obtained, for example, in the hydrogenation of technical fatty acid methyl esters or in the hydrogenation of aldehydes from Roelen's oxosynthesis. Alkyl oligoglucosides having a chain length of C₈ to C₁₀ (DP=1 to 3), which are obtained as first runnings in the separation of technical C₈₋₁₈ coconut oil fatty alcohol by distillation and which may contain less than 6% by weight of C₁₂ alcohol as an im-purity, and also alkyl oligoglucosides based on technical C_{9/11} oxoalcohols (DP=1 to 3) are preferred. In addition, the alkyl or alkenyl radical R⁹ may also be derived from primary alcohols containing 12 to 22 and preferably 12 to 14 carbon atoms. Typical examples are lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and technical mixtures thereof which may be obtained as described above. Alkyl oligo-glucosides based on hydrogenated C_{12/14} cocoalcohol with a DP of 1 to 3 are preferred. Alkyl and/or alkenyl oligoglycosides are used in quantities of 1 to 90% by weight, preferably in quantities of 5 to 70% by weight and more particularly in quantities of 10 to 50% by weight in the preparations according to the invention.

Fatty Alcohols

Fatty alcohols are understood to be primary aliphatic alcohols corresponding to formula (II):
R²OH (11)
where R² is an aliphatic, linear or branched hydrocarbon radical containing 6 to 22 carbon atoms and 0 and/or 1, 2 or 3 double bonds. Typical examples are caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof obtained, for example, in the high-pressure hydrogenation of technical methyl esters based on fats and oils or aldehydes from Roelen's oxo synthesis and as monomer fraction in the dimerization of unsaturated fatty alcohols. Preferred fatty alcohols are technical C₁₂₋₁₈ fatty alcohols such as, for example, coconut oil, palm oil, palm kernel oil or tallow fatty alcohol. In order not to disturb the balance between cleaning effect and lipid-layer-enhancing effect, fatty alcohols containing 12 to 14 carbon atoms are particularly preferred.

These fatty alcohols are used in the preparations according to the invention in quantities of 0.1 to 10% by weight, preferably in quantities of 0.2 to 8% by weight and more particularly in quantities of 0.5 to 5% by weight.

Commercial Applications

The preparations according to the invention are distinguished by a high cleaning capacity and by excellent lipid-layer-enhancing properties. They show high dermatological compatibility, are liquid and pumpable and, where fatty alcohols are added, can be produced without heat. They have thickening properties and are completely biodegradable.

Accordingly, the present invention also relates to the use of fruit acid esters as lipid layer enhancers in surfactant-containing formulations, for example for hair and body care.

Embodiments of the lipid-layer-enhancing preparations according to the invention contain
a) 0.1 to 30% by weight fruit acid esters,
b) 1 to 90% by weight anionic and/or nonionic and/or cationic and/or amphoteric and/or zwitterionic surfactants and
c) 0.1 to 10% by weight fatty alcohols,
   preferably
a) 0.5 to 25% by weight fruit acid esters,
b) 5 to 70% by weight anionic and/or nonionic and/or cationic and/or amphoteric and/or zwitterionic surfactants and
c) 0.2 to 8% by weight fatty alcohols,
   more preferably
a) 1 to 20% by weight fruit acid esters,
b) 10 to 50% by weight anionic and/or nonionic and/or cationic and/or amphoteric and/or zwitterionic surfactants and
c) 0.5 to 5% by weight fatty alcohols,
   most preferably
a) 0.5 to 25% by weight fruit acid esters,
b) 5 to 70% by weight anionic and/or nonionic and/or cationic and/or amphoteric and/or zwitterionic surfactants and
c) 0.5 to 5% by weight fatty alcohols,
   with the proviso that the quantities shows add up to 100% by weight with water and optionally typical auxiliaries and additives.

The cosmetic and/or pharmaceutical preparations mentioned, such as for example hair shampoos, shower baths, foam baths, washing lotions and the like, may contain emulsifiers, pearlizing waxes, consistency factors, thickeners, polymers, silicone compounds, lecithins, phospholipids, biogenic agents, antioxidants, antidandruff agents, film formers, hydrotropes, solubilizers, preservatives, perfume oils, dyes and the like as further auxiliaries and additives.

Emulsifiers

Suitable emulsifiers are, for example, nonionic surfactants from at least one of the following groups:
products of the addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and alkylamines containing 8 to 22 carbon atoms in the alkyl group; >addition products of 15 to 60 mol ethylene oxide onto castor oil and/or hydrogenated castor oil;
partial esters of glycerol and sorbitan with unsaturated, linear or saturated, branched fatty acids containing 12 to 22 carbon atoms and/or hydroxycarboxylic acids containing 3 to 18 carbon atoms and addition products thereof onto 1 to 30 mol ethylene oxide;
partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and addition products thereof onto 1 to 30 mol ethylene oxide;
mixed esters of pentaerythritol, fatty acids, citric acid and fatty alcohol and/or mixed esters of fatty acids containing 6 to 22 carbon atoms, methyl glucose and polyols, preferably glycerol or polyglycerol,
mono-, di- and trialkyl phosphates and mono-, di- and/or tri-PEG-alkyl phosphates and salts thereof,
wool wax alcohols,
polysiloxane/polyalkyl/polyether copolymers and corresponding derivatives,
block copolymers, for example Polyethylene glycol-30 Dipolyhydroxystearate;
polymer emulsifiers, for example Pemulen types (TR-1, TR-2) of Goodrich;
polyalkylene glycols and
glycerol carbonate.
   Ethylene Oxide Addition Products

The addition products of ethylene oxide and/or propylene oxide onto fatty alcohols, fatty acids, alkylphenols or onto castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of adducts of ethylene oxide with glycerol are known as lipid layer enhancers for cosmetic formulations.

Partial Glycerides

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide with the partial glycerides mentioned are also suitable.

Sorbitan Esters

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Anionic Emulsifiers

Typical anionic emulsifiers are aliphatic fatty acids containing 12 to 22 carbon atoms, such as for example palmitic acid, stearic acid or behenic acid, and dicarboxylic acids containing 12 to 22 carbon atoms, such as azelaic or sebacic acid for example.

Amphoteric and Cationic Emulsifiers

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipro-pionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-coco-alkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polymers, polyvinyl alcohol and polyvinyl pyrrolidone. Other consistency factors which have proved to be particularly effective are bentonites, for example Bentone® Gel VS-5PC (Rheox) which is a mixture of cyclopentasiloxane, Disteardimonium Hectorite and propylene carbonate. Other suitable consistency factors are surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat poly-peptides, polyethyleneimine, cationic silicone polymers such as, for example, amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar®)CBS, Jaguar®)C-17, Jaguar®C-16 of Celanese, quaternized ammonium salt polymers such as, for example, Mirapol®) A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamido-propyl trimethylammonium chloride/acrylate copolymers, octylacryl-amide/methyl methacrylate/tert.-butylaminoethyl methacrylate/2-hydroxy-propyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

Silicone Compounds Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates.

Biogenic Agents

Biogenic agents in the context of the invention are, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example prunus extract and bambara nut extract, and vitamin complexes.

Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Antidandruff Agents

Suitable antidandruff agents are Pirocton Olamin (1-hydroxy-4-methyl-6-(2,4,4-trimethylpentyl)-2-(1H)-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxy-phenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 dalton;
technical oligoglycerol mixtures with a degree of self-condensation of 1.5 to 10 such as, for example, technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine;
dialcoholamines, such as diethanolamine or 2-aminopropane-1,3-diol.
   Preservatives

Suitable preservatives are, for example, phenoxyethanol, formaldehyde solution, parabens, pentanediol or sorbic acid and the silver complexes known under the name of Surfacine® and the other classes of compounds listed in Appendix 6, Parts A and B of the Kosmetikverordnung (“Cosmetics Directive”).

Perfume Oils and Aromas

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat. Suitable aromas are, for example, peppermint oil, spearmint oil, aniseed oil, Japanese anise oil, caraway oil, eucalyptus oil, fennel oil, citrus oil, wintergreen oil, clove oil, menthol and the like.

Dyes

Suitable dyes are any of the substances suitable and approved for cosmetic purposes. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS(C.I. 69800) and madder lake (C.I. 58000). Luminol may also be present as a luminescent dye. These dyes are normally used in concentrations of 0.001 to 0.1% by weight, based on the mixture as a whole.

### EXAMPLES

### I. Formulation Examples

Quantities in % by weight, viscosity measurement: Brookfield RVT, spindle 4 at 10 r.p.m./RT (23±2° C.)

TABLE 1a
Preparations for liquid processing (percentages as %
by weight) - appearance cloudy

| Trade Name | INCI Name | 1 | 2 | 3 |
|---|---|---|---|---|
| Cutina ® | Citric acid | 25.0 | 25.0 | 20.0 |
| KE 3602 | trioctadecyl ester | | | |
| Texapon ® | Sodium Laureth | — | 20.0 | — |
| NSO | Sulfate | | | |
| Plantacare ® | Laurylglycoside | 20.0 | — | — |
| 1200 UP | | | | |
| Dehydol ® | Fatty alcohol | 5.0 | 5.0 | — |
| LS 4 DEO | C₁₂₋₁₄ + 4 EO | | | |
| Dehyton ® | Cocamidopropyl | 20.0 | — | — |
| PK 45 | Betaine | | | |
| Dehyton ® | Sodium | — | — | 77.45 |
| MC | Cocoamphoacetate | | | |
| Lorol ® C18 | Fatty alcohol C₁₈ | 0.20 | 0.20 | 0.20 |
| Citric acid 50% | | 1.5 | 0.2 | 2.0 |
| Benzoic acid | | 0.35 | 0.35 | 0.35 |
| Water | | to 100.0 | to 100.0 | to 100.0 |
| pH value | | 4.2 | 3.3 | 4.5 |
| Viscosity | | 7,700 | 9,600 | 12,000 |
| [mPa * s] | | | | |

TABLE 1b
Preparations for liquid processing (percentages as %
by weight) - appearance cloudy

| Trade Name | INCI Name | 4 | 5 | 6 |
|---|---|---|---|---|
| Cutina ® | Malic acid | 25.0 | 25.0 | 20.0 |
| KE 3580 | dioctadecyl ester | | | |
| Texapon ® | Sodium Laureth | — | 20.0 | — |
| NSO | Sulfate | | | |
| Plantacare ® | Laurylglycoside | 20.0 | — | — |
| 1200 UP | | | | |
| Dehydol ® LS | Fatty alcohol | 5.0 | 5.0 | — |
| 4 DEO | C₁₂₋₁₄ + 4 EO | | | |
| Dehyton ® | Cocamidopropyl | 20.0 | — | — |
| PK 45 | Betaine | | | |
| Dehyton ® | Sodium | — | — | 77.45 |
| MC | Cocoamphoacetate | | | |
| Lorol ® C18 | Fatty alcohol C₁₈ | 0.20 | 0.20 | 0.20 |
| Citric acid 50% | | 1.5 | 0.2 | 2.0 |
| Benzoic acid | | 0.35 | 0.35 | 0.35 |
| Water | | to 100.0 | to 100.0 | to 100.0 |
| pH value | | 4.2 | 3.3 | 4.5 |
| Viscosity | | 7,100 | 9,500 | 11,800 |
| [mPa * s] | | | | |

TABLE 2a
Preparations for liquid processing (percentages
as % by weight) - gel-like, appearance clear

| Trade Name | INCI Name | 7 | 8 | 9 |
|---|---|---|---|---|
| Cutina ® | Citric acid | 20.0 | 23.0 | 20.0 |
| KE 3602 | trioctadecyl ester | | | |
| Eutanol G | 2-Octyl-dodecanol | — | — | 4.0 |
| Glycerol | | 5.0 | 15.0 | 10.0 |
| Texapon ® | Sodium Laureth | — | 5.0 | — |
| NSO | Sulfate | | | |
| Plantacare ® | Laurylglycoside | 28.0 | — | 15.0 |
| 1200 UP | | | | |
| Dehydol ® | Fatty alcohol | — | 15.0 | — |
| LS 4 DEO | C₁₂₋₁₄ + 4 EO | | | |
| Dehyton ® | Cocamidopropyl | — | — | 15.0 |
| PK 45 | Betaine | | | |
| Dehyton ® | Sodium | — | — | 5.0 |
| MC | Cocoamphoacetate | | | |
| Lorol ® C18 | Fatty alcohol C₁₈ | 0.20 | 0.20 | 0.20 |
| Citric acid 50% | | 3.5 | 0.5 | 0.5 |
| Benzoic acid | | 0.35 | 0.35 | 0.35 |
| Water | | to 100.0 | to 100.0 | to 100.0 |
| pH value | | 4.2 | 3.3 | 4.5 |
| Viscosity | | 25,500 | 33,100 | 38,800 |
| [mPa * s] | | | | |

TABLE 2b
Preparations for liquid processing (percentages
as % by weight) - gel-like,appearance clear

| Trade Name | INCI Name | 10 | 11 | 12 |
|---|---|---|---|---|
| Cutina ® KE 3580 | Malic acid | 20.0 | 23.0 | 20.0 |
| | dioctadecyl ester | | | |
| Eutanol G | 2-Octyl-dodecanol | — | — | 4.0 |
| Glycerol | | 5.0 | 15.0 | 10.0 |
| Texapon ® | Sodium Laureth | — | 5.0 | — |
| NSO | Sulfate | | | |
| Plantacare ® | Laurylglycoside | 28.0 | — | 15.0 |
| 1200 UP | | | | |
| Dehydol ® | Fatty alcohol | — | 5.0 | — |
| LS 4 DEO | C₁₂₋₁₄ + 4 EO | | | |
| Dehyton ® | Cocamidopropyl | — | — | 15.0 |
| PK 45 | Betaine | | | |
| Dehyton ® | Sodium | — | — | 5.0 |
| MC | Cocoamphoacetate | | | |
| Lorol ® C18 | Fatty alcohol C₁₈ | 0.20 | 0.20 | 0.20 |
| Citric acid 50% | | 1.5 | 0.2 | 2.0 |
| Benzoic acid | | 0.35 | 0.35 | 0.35 |
| Water | | to 100.0 | to 100.0 | to 100.0 |
| pH value | | 4.2 | 3.3 | 4.5 |
| Viscosity | | 25,200 | 31,800 | 39,600 |
| [mPa * s] | | | | |

TABLE 3
Incorporation of the fruit wax dispersion in shampoo and shower
bath formulations(quantities in % by weight)

| Trade Name | INCI Name | C1 | 1 | 2 |
|---|---|---|---|---|
| Texapon ® NSO | Sodium Laureth Sulfate | 35.0 | 35.0 | 35.0 |
| Dehyton ® MC | Sodium Cocoamphoacetate | 8.0 | — | — |
| Wax-containing compound formulation 3, Table 1a | | — | 10.0 | — |
| Wax-containing compound formulation 6, Table 1b | | — | — | 10.0 |
| Cosmedia Guar ® C 261 N | Guar Gum | 0.2 | 0.2 | 0.2 |
| Perfume oil | | 0.3 | 0.3 | 0.3 |
| Euxyl ® K | Preservative | 0.1 | 0.1 | 0.1 |
| Sodium chloride | | 0.85 | 1.2 | 0.95 |
| Water | | to 100.0 | to 100.0 | to 100.0 |
| Citric acid | for pH adjustment | | | |
| pH value | | 5.5 | 5.4 | 5.4 |
| Viscosity [mPa * s] | | 5,100 | 5,500 | 6,000 |
| Sensory Assessment | | | | |
| Hair | Combability | ⋄ | ++ | +++ |
| | Stress | ⋄ | +++ | ++ |
| | Electrostatic charging | − | +++ | ++ |
| | Manageability | −− | ++ | +++ |
| Skin | Foam creaminess | ⋄ | +++ | ++ |
| | Skin feel | + | ++ | ++ |
| | Moisture | − | +++ | ++ |
| | TEWL [mg/cm²/n] | 4.1 | 1.2 | 1.4 |

Properties:
(−−) very poor,
(−) poor,
(⋄) neutral,
(+) good,
(++) very good,
(+++) exceptionally good
TEWL values:
(>6) poor,
(6-4) normal,
(2-4) good,
(<2) very good

1-11. (cancelled).

12. A cosmetic and/or pharmaceutical composition comprising:
(a) a fruit acid ester;
(b) a surfactant selected from the group consisting of an anionic surfactant, a nonionic surfactant, a cationic surfactant, an amphoteric surfactant, a zwitterionic surfactant, and mixtures thereof;
(c) a fatty alcohol; and
(d) optionally, water.

13. The composition of claim 12 wherein (a) is present in the composition in an amount of from about 0.1 to 30% by weight, (b) is present in an amount of from about 0.1 to 90% by weight, and (c) is present in an amount of from about 0.1 to 10% by weight, all weights being based on the weight of the composition.

14. The composition of claim 12 wherein (a) is present in the composition in an amount of from about 0.5 to 25% by weight, (b) is present in the composition in an amount of from about 5 to 70% by weight, and (c) is present in the composition in an amount of from about 0.2 to 8% by weight, all weights being based on the total weight of the composition.

15. The composition of claim 12 wherein the composition is capable of being cold processed.

16. The composition of claim 12 wherein the acid component of (a) is selected from the group consisting of citric acid, malic acid, tartaric acid, mandelic acid, racemic acid, and mixtures thereof.

17. The composition of claim 12 wherein (a) is a fruit acid alkyl ester selected from the group consisting of a fruit acid monoalkyl ester, a fruit acid dialkyl ester, a fruit acid trialkyl ester, and mixtures thereof.

18. The composition of claim 12 wherein the alcohol component of (a) is a C₈₋₂₂ alkanol.

19. The composition of claim 12 wherein (c) is a C₁₀₋₁₈ fatty alcohol.

20. The composition of claim 12 wherein (b) is an alkyl and/or alkenyl oligoglycoside.

21. A process for treating human skin and/or hair comprising contacting the skin and/or hair with a composition containing:
(a) a fruit acid ester;
(b) a surfactant selected from the group consisting of an anionic surfactant, a nonionic surfactant, a cationic surfactant, an amphoteric surfactant, a zwitterionic surfactant, and mixtures thereof;
(c) a fatty alcohol; and
(d) optionally, water.

22. The process of claim 21 wherein (a) is present in the composition in an amount of from about 0.1 to 30% by weight, (b) is present in an amount of from about 0.1 to 90% by weight, and (c) is present in an amount of from about 0.1 to 10% by weight, all weights being based on the weight of the composition.

23. The process of claim 21 wherein (a) is present in the composition in an amount of from about 0.5 to 25% by weight, (b) is present in the composition in an amount of from about 5 to 70% by weight, and (c) is present in the composition in an amount of from about 0.2 to 8% by weight, all weights being based on the total weight of the composition.

24. The process of claim 21 wherein the composition is capable of being cold processed.

25. The process of claim 21 wherein the acid component of (a) is selected from the group consisting of citric acid, malic acid, tartaric acid, mandelic acid, racemic acid, and mixtures thereof.

26. The process of claim 21 wherein (a) is a fruit acid alkyl ester selected from the group consisting of a fruit acid monoalkyl ester, a fruit acid dialkyl ester, a fruit acid trialkyl ester, and mixtures thereof.

27. The process of claim 21 wherein the alcohol component of (a) is a C₈₋₂₂ alkanol.

28. The process of claim 21 wherein (c) is a C₁₀₋₁₈ fatty alcohol.

29. The process of claim 21 wherein (b) is an alkyl and/or alkenyl oligoglycoside.

30. A process for enhancing the lipid-layer effect of a surfactant-containing composition comprising adding to the composition a fruit acid (alkyl) ester.
